(19) United States
(12) Patent Application Publication
(10) Pub. No.: US 2014/0296969 A1
(43) Pub. Date: Oct. 2, 2014

(54) Anterior Leaflet Clip Device for Prosthetic Mitral Valve

(71) Applicant: Tendyne Holdings, Inc., Roseville, MN (US)

(72) Inventors: Zachary J. TEGELS, Minneapolis, MN (US); Craig A. EKVALL, Bethel, MN (US); Robert M. VIDLUND, Forest Lake, MN (US)

(73) Assignee: TENDYNE HOLDLINGS, INC., Roseville, MN (US)

(21) Appl. No.: 14/154,546

(22) Filed: Jan. 14, 2014

Related U.S. Application Data
(60) Provisional application No. 61/807,695, filed on Apr. 2, 2013.

(51) Int. Cl. A61F 2/24

(52) U.S. Cl. CPC A61F 2/2418; A61F 2/2427
USPC 623/2.11; 623/2.18

(57) ABSTRACT

This invention relates to a pre-configured compressible transcatheter prosthetic cardiovascular valve having an improved anterior leaflet clip device. The invention also relates to methods for deploying such a valve for treatment of a patient in need thereof.

### CROSS REFERENCE TO RELATED APPLICATIONS

Not applicable

### STATEMENT REGARDING FEDERALLY SPONSORED RESEARCH OR DEVELOPMENT

No federal government funds were used in researching or developing this invention.

### NAMES OF PARTIES TO A JOINT RESEARCH AGREEMENT

Not applicable.

### SEQUENCE LISTING INCLUDED AND INCORPORATED BY REFERENCE HEREIN

Not applicable.

### BACKGROUND

1. Field of the Invention

This invention relates to an improved transcatheter prosthetic heart valve that comprises an anterior leaflet clip device for reducing or preventing leaking around an implanted self-expanding stent and valve assembly that is anchored within the mitral valve or triscuspid valve of the heart using an optional integral cuff to anchor the valve and using one or more tethers anchored to the heart, and a delivery system therefor.

2. Background of the Invention

Valvular heart disease and specifically aortic and mitral valve disease is a significant health issue in the US. Annually approximately 90,000 valve replacements are conducted in the US. Traditional valve replacement surgery, the orthotopic replacement of a heart valve, is an “open heart” surgical procedure. Briefly, the procedure necessitates surgical opening of the thorax, the initiation of extra-corporeal circulation with a heart-lung machine, stopping and opening the heart, excision and replacement of the diseased valve, and re-starting of the heart. While valve replacement surgery typically carries a 1-4% mortality risk in otherwise healthy persons, a significantly higher morbidity is associated to the procedure largely due to the necessity for extra-corporeal circulation. Further, open heart surgery is often poorly tolerated in elderly patients.

Thus if the extra-corporeal component of the procedure could be eliminated, morbidities and cost of valve replacement therapies would be significantly reduced.

While replacement of the aortic valve in a transcatheter manner is the subject of intense investigation, lesser attention has been focused on the mitral valve. This is in part reflective of the greater level of complexity associated to the native mitral valve apparatus and thus a greater level of difficulty with regards to inserting and anchoring the replacement prosthesis.

Several designs for catheter-deployed (transcatheter) aortic valve replacement are under various stages of development. The Edwards SAPIEN® transcatheter heart valve is currently undergoing clinical trial in patients with calcific aortic valve disease who are considered high-risk for conventional open-heart valve surgery. This valve is deployable via a retrograde transarterial (transfemoral) approach or an antegrade transapical (transventricular) approach. A key aspect of the Edwards SAPIEN® and other transcatheter aortic valve replacement designs is their dependence on lateral fixation (e.g., tines) that engages the valve tissues as the primary anchoring mechanism. Such a design basically relies on circumferential friction around the valve housing or stent to prevent dislodgement during the cardiac cycle. This anchoring mechanism is facilitated by, and may somewhat depend on, a calcified aortic valve annulus. This design also requires that the valve housing or stent have a certain degree of rigidity.

At least one transcatheter mitral valve design is currently in development. The Endovalve uses a folding tripod-like design that delivers a tri-leaflet bioprosthetic valve. It is designed to be deployed from a minimally invasive transatrial approach, and could eventually be adapted to a transvenous atrial septotomy delivery. This design uses “proprietary gripping features” designed to engage the valve annulus and leaflets tissues. Thus the anchoring mechanism of this device is essentially equivalent to that used by transcatheter aortic valve replacement designs.

Various problems continue to exist in this field, including problems with insufficient articulation and sealing of the valve within the native annulus, pulmonary edema due to poor atrial drainage, perivalvular leaking around the install prosthetic valve, lack of a good fit for the prosthetic valve within the native mitral annulus, atrial tissue erosion, excess wear on the nitinol structures, interference with the aorta at the posterior side of the mitral annulus, and lack of customization, to name a few. Accordingly, there is still a need for an improved prosthetic mitral valve having a commissural sealing structure.

### BRIEF SUMMARY OF THE INVENTION

The present invention relates to the improved design and function of novel pre-configured compressible transcatheter prosthetic cardiovascular valves having an anterior leaflet clip device for reducing or preventing leaking around an implanted self-expanding stent and valve assembly that is anchored within the mitral valve of the heart.

In a preferred embodiment, there is provided a pre-configured compressible transcatheter prosthetic cardiovascular valve having an anterior leaflet clip device, which comprises an expandable leaflet assembly comprised of stabilized tissue or synthetic material disposed within an expandable stent having at a distal end a plurality of articulating collar support structures having a tissue covering to form an atrial collar, said expandable stent having a proximal end comprised of an integral tether connection apparatus, said anterior leaflet clip device comprising a folding component that is mounted on the expandable stent, said folding component having a hinge segment and a leaflet capture and attachment segment.

In a preferred embodiment, the anterior leaflet clip device is an integrated component of the expandable stent.

In another preferred embodiment, the anterior leaflet clip device is a separate component that is sutured to the expanded stent before deployment of the valve, the device having a mounting segment connected to the hinge segment which is connected to the leaflet capture and attachment segment.

In a preferred embodiment, the anterior leaflet clip further comprises a plurality of pre-aligned suture holes in the leaflet capture and attachment segment.

In a preferred embodiment, the pre-aligned suture holes in the mounting segment and the leaflet capture and attachment segment wherein the suture holes line up in a substantially adjacent manner after the leaflet capture and attachment segment is folded over.

The design as provided focuses on the deployment of a device via a minimally invasive fashion and by way of example considers a minimally invasive surgical procedure utilizing the intercostal or subxyphoid space for valve introduction, but may also include standard retrograde, or antegrade transcatheter approaches. In order to accomplish this, the valve is formed in such a manner that it can be compressed to fit within a delivery system and secondarily ejected from the delivery system into the target location, for example the mitral or tricuspid valve annulus.

Wire-Frame Commissural Sealing Structure with Stent Variations

In a preferred embodiment, there is provided a prosthetic mitral valve containing an expandable leaflet assembly comprised of stabilized tissue or synthetic material disposed within a self-expanding stent having a flared collar at its distal end and anterior leaflet clip device attached under the collar and forming an anterior leaflet sealing shell.

In another preferred embodiment, there is provided a prosthetic heart valve as described having a single tether connecting the proximal end of the stent to an epicardial securing device at or near the apex of the left ventricle. In another preferred embodiment, the prosthetic mitral valve does not use an anchoring or positioning tether at all, and instead is held in the mitral annulus by the wrapping forces of the native leaflets, and optionally one or more standard anchoring elements, including but not limited to barbs, pins, and/or hooks, or combinations thereof.

In another preferred embodiment, there is provided a prosthetic heart valve as described, wherein the anterior leaflet sealing device comprises a partial seal at a specific location such as designated for an A2 leaflet segment of a mitral valve, or a full circumferential ring seal attached to and hanging below the collar, wherein the sealing component may also have a sub-annular shape in an oval, with a short diameter from posterior-to-anterior about the same diameter as the stent body, about 24 mm, and a long diameter from commissure-to-commissure of about 32 mm. These dimensions will necessarily vary depending on patient needs, and are given only as non-limiting examples based upon anatomical averages.

In another preferred embodiment, there is provided a prosthetic cardiovascular valve with a stent body that has a low height to width profile.

In a preferred embodiment, the prosthetic mitral valve contains an improved stent body that is a half-round D-shape in cross-section.

In a preferred embodiment, the prosthetic mitral valve contains an improved stent body that is a bent tubular stent structure wherein the bend is directed away from the anterior leaflet, away from interfering with coaptation of adjacent (e.g., aortic) valvular leaflets.

In a preferred embodiment, the prosthetic mitral valve contains an improved stent body that has a low height to width profile and the leaflet structure disposed within the stent is positioned at or near the atrial end of the stent body.

In another preferred embodiment, the a prosthetic mitral valve has a stent body made from both braided wire (atrial end) and laser-cut metal (annular or ventricular end), or vice versa.

Additional Features for Improved Stents

In a preferred embodiment, the prosthetic heart valve has a cuff that has articulating wire articulating radial tines or posts of wire of various lengths.

In another preferred embodiment, the prosthetic heart valve has at least one elastic tether to provide compliance during the physiologic movement or conformational changes associated with heart contraction.

In another preferred embodiment, the prosthetic heart valve has a stent body and cuff that are made from a superelastic metal.

In another preferred embodiment, the prosthetic heart valve has a tether which is used to position the valve cuff into the mitral annulus to prevent perivalvular leak.

In another preferred embodiment, the tethers are bioabsorbable and provide temporary anchoring until biological fixation of the prosthesis occurs. Biological fixation, as contemplated herein, consists of fibrous adhesions between the leaflet tissues and prosthesis or compression on the prosthesis by reversal of heart dilation, or both.

In another preferred embodiment, the prosthetic heart valve has a cuff for a prosthetic heart valve, said cuff being covered with tissue.

In another preferred embodiment, the cuff is covered with a synthetic polymer selected from expandable polytetrafluoroethylene (ePTFE) or polyester.

In another preferred embodiment, there is provided a prosthetic heart valve that has leaflet material constructed from a material selected from the group consisting of polyurethane, polytetrafluoroethylene, pericardium, and small intestine submucosa.

In another preferred embodiment, there is provided a prosthetic heart valve having surfaces that are treated with anticoagulant.

In another preferred embodiment, there is provided a prosthetic heart valve having a cuff and containing anchoring tethers which are attached to the cuff.

In another preferred embodiment, there is provided a prosthetic heart valve having a cuff and containing anchoring tethers which are attached to the cuff and at both commissural tips.

In another preferred embodiment, there is provided a prosthetic heart valve having a cuff where the cuff attachment relative to the body is within the angles of about 60 degrees to about 150 degrees.

In another preferred embodiment, there is provided a prosthetic heart valve containing a combination of tethers and barbs useful for anchoring the device into the mitral annulus.

In another embodiment, the wire of the cuff is formed as a series of radially extending articulating radial tines or posts of wire of equal or variable length.

In another embodiment, the cuff extends laterally beyond the expanded tubular stent according to a ratio of the relationship between the height of the expanded deployed stent (h) and the lateral distance that the cuff extends onto the tissue (l). Preferably, the h/l ratio can range from 1:10 to 10:1, and more preferably includes without limitation 1:3, 1:2, 1:1, 2:1, and fractional ranges there between such as 1.25:2.0, 1.5:2.0, and so forth. It is contemplated in one non-limiting example that the cuff can extend laterally (l) between about 3 and about 30 millimeters.

In another embodiment, there is provided a feature wherein the tubular stent has a first end and a second end, wherein the cuff is formed from the stent itself, or in the alternative is formed separately and wherein the cuff is located at the first end of the stent, and the second end of the tubular stent has a plurality of tether attachment structures.

In another embodiment, there is provided a feature further comprising a plurality of tethers for anchoring the prosthetic heart valve to tissue and/or for positioning the prosthetic heart valve.

In another embodiment, there is provided a feature further comprising an epicardial tether securing device, wherein the tethers extend from about 2 cm to about 20 cm in length, and are fastened to an epicardial tether securing device. Some pathological conditions within a ventricle may require a atrial-apical tether from about 8 to about 15 cm or more, as described within the range above.

In another embodiment, there is provided a catheter delivery system for delivery of a prosthetic heart valve which comprises a delivery catheter having the prosthetic heart valve disposed therein, and an obturator for expelling the prosthetic heart valve.

In another embodiment, there is provided an assembly kit for preparing the catheter delivery system which comprises a compression funnel, an introducer, a wire snare, an obturator, a delivery catheter, and a prosthetic heart valve, wherein the compression funnel has an aperture for attaching to the introducer, wherein said introducer is comprised of a tube having a diameter that fits within the diameter of the delivery catheter, wherein said obturator is comprised of a tube fitted with a handle at one end and a cap at the other end, wherein said cap has an opening to allow the wire snare to travel through, and said obturator has a diameter that fits within the diameter of the introducer, and wherein said prosthetic heart valve is compressible and fits within the delivery catheter.

In another embodiment, there is provided a method of treating mitral regurgitation and/or tricuspid regurgitation in a patient, which comprises the step of surgically deploying the prosthetic heart valve described herein into the annulus of the target valve structure, e.g. mitral valve annulus and tricuspid valve annulus of the patient.

In another embodiment, there is provided a feature wherein the prosthetic heart valve is deployed by directly accessing the heart through an intercostal space, using an apical approach to enter the left (or right) ventricle, and deploying the prosthetic heart valve into the valvular annulus using the catheter delivery system.

In another embodiment, there is provided a feature wherein the prosthetic heart valve is deployed by directly accessing the heart through a thoracotomy, sternotomy, or minimally-invasive thoracic, thorascopic, or transdiaphragmatic approach to enter the left (or right) ventricle, and deploying the prosthetic heart valve into the valvular annulus using the catheter delivery system.

In another embodiment, there is provided a feature wherein the prosthetic heart valve is deployed by directly accessing the heart through the intercostal space, using a lateral approach to enter the left or right ventricle, and deploying the prosthetic heart valve into the valvular annulus using the catheter delivery system.

In another embodiment, there is provided a feature wherein the prosthetic heart valve is deployed by accessing the left heart using either an antegrade-trans(atrial)septal (transvenous-trans(atrial)septal) approach or a retrograde (transarterial-transaortic) catheter approach to enter the left heart, and deploying the prosthetic heart valve into the mitral annulus using the catheter delivery system.

In another embodiment, there is provided a feature wherein the prosthetic heart valve is deployed into the mitral annulus from a retrograde approach by accessing the left ventricle through the apex of the ventricular septum (transvenous-trans(ventricular)septal approach).

In another embodiment, there is a feature wherein the prosthetic heart valve is deployed into the mitral position using a retrograde transventricular septal approach and the tethers are anchored into or on the right ventricular side of the ventricular septum.

In another embodiment, there is provided a feature further comprising tethering the prosthetic heart valve to tissue within the left ventricle.

In another embodiment, there is provided a feature wherein the prosthetic heart valve is tethered to the apex of the left ventricle using an epicardial tether securing device.

In another embodiment, there is provided a retrieval method for quickly removing a prosthetic heart valve having one or more tethers from a patient using minimally invasive cardiac catheter techniques, which comprises the steps of: capturing the one or more tethers with a catheter having a snare attachment, guiding the captured tethers into a collapsible funnel attachment connected to the removal catheter, pulling the tethers to conform the prosthetic heart valve into a collapsed, compressed conformation, and pulling the now compressed prosthetic heart valve into the removal catheter for subsequent extraction. The retrieval method is contemplated for use for capturing the prosthetic heart valve as described herein or any suitable tethered, collapsible medical device. In a preferred embodiment, the method is used to extract a prosthetic heart valve from either the left or right ventricle. The method may be particularly useful to extract the prosthetic appliance during an aborted surgical deployment.

Articulating Collar Support Structures with Collar Variations

In another preferred embodiment, there is provided a method of sealing a deployed prosthetic mitral valve against hemodynamic leaking, comprising fitting a prosthetic mitral valve with a flared end or cuff having articulating collar support structures prior to deployment wherein the flared end or cuff is constructed to contour to the commissures of a pathologically defective mitral valve and constructed to contour to the zone of coaptation of the pathologically defective mitral valve, wherein the flared end or cuff is formed from wire originating from one end of an expandable tubular braided wire stent and the flared end or cuff is covered with stabilized tissue or synthetic material, the commissural contour components of the flared end or cuff and the zone of coaptation contour components of the flared end or cuff forming a complete or partial saddle-shape wherein the commissural contour components are in direct communication with the mitral valve commissures, and the zone of coaptation contour components are in direct communication with the mitral valve zone of coaptation.

In a preferred embodiment, the flared end or cuff shape is D-shaped, agaricoid, onychoid, reniform, an oval, a truncated-oval having a squared end, propeller-shaped having two or three blades, cruciform, petal-shaped having flat radial covered articulating radial tines or posts of wire, irregular or amoeboid, cotyloid shaped, partial half-round fan-shape, rectangular U-shape.

In another preferred embodiment, the flared end or cuff is constructed from ductile metal.

In another preferred embodiment, the flared end or cuff shape is constructed with a cover of stabilized tissue that is derived from adult, or 90-day old, or 30-day old bovine, ovine, equine or porcine pericardium, or from animal small intestine submucosa.

In another preferred embodiment, the flared end or cuff shape is constructed with a cover of synthetic material selected from the group consisting of polyester, polyurethane, and polytetrafluoroethylene.

In another preferred embodiment, the flared end or cuff shape is constructed with a cover of another suitable elastomeric material.

In another preferred embodiment, the elastomeric material, silicone, stabilized tissue or synthetic material is treated with anticoagulant.

In another preferred embodiment, the method further comprises the step of anchoring the prosthetic heart valve to tissue uses a plurality of tethers to the flared end or cuff.

In another preferred embodiment, the method further comprises the step of anchoring the prosthetic heart valve to tissue using a single tether attached to the stent or a tether-attachment structure attached to the stent.

In another preferred embodiment, at least one of the plurality of tethers is an elastic tether.

In another preferred embodiment, at least one of the plurality of tethers is a bioresorbable tether.

### BRIEF DESCRIPTION OF THE DRAWINGS

FIG. 1 is a side-view of one preferred type of anterior leaflet clip device.

FIG. 2 is a side view of the anterior leaflet clip device of FIG. 1 clipped onto a native anterior leaflet.

FIG. 3 is a side-view of another preferred type of anterior leaflet clip device.

FIG. 4 is a side view of the anterior leaflet clip device of FIG. 3 clipped onto a native anterior leaflet.

FIG. 5 is a side-view of another preferred type of anterior leaflet clip device.

FIG. 6 is a side view of the anterior leaflet clip device of FIG. 5 clipped onto a native anterior leaflet.

FIG. 7 is a perspective anterolateral view of one embodiment showing laser cut stent with valve leaflets mounted therein and with tissue-covered articulating collar structure attached, and the anterior leaflet clip device attached to the stent and/or collar, and providing an attachment mechanism to secure the anterior leaflet and provide a subvalvular seal against retrograde hemodynamic forces.

FIG. 8a is a side view of one embodiment showing laser cut stent with valve leaflets mounted therein (not shown) and with tissue-covered articulating collar structure attached, and the anterior leaflet clip device attached to the stent, and providing an attachment mechanism to secure the anterior leaflet and provide a subvalvular seal against retrograde hemodynamic forces.

FIG. 8b is a side view of one embodiment showing the leaflet clip sutured across the anterior leaflet and securing it to the laser cut stent body, providing an attachment mechanism to secure the anterior leaflet and provide a subvalvular seal against retrograde hemodynamic forces.

FIG. 9 is a side view of one embodiment showing the leaflet clip manufactured as an integral component of the laser-cut stent body. FIG. 9 shows how the clip moves from the first deployment position to the second leaflet capture position.

### DETAILED DESCRIPTION OF THE INVENTION

During systole the anterior leaflet of the mitral valve forms a semilunar seal with the posterior leaflet, and during diastole the anterior leaflet separates from the posterior leaflet to accommodate the emptying flow of the left atrium into the left ventricle. When the mitral valve is insufficient, regurgitant jets are directed back into the left atrium during systole. Deployment of a prosthetic mitral valve is one suitable solution to address this problem. Where a prosthetic mitral valve is deployed directly into the native annulus, the anterior and posterior leaflets are separated and form a partial seal around the circumference of the valve body. However, complete or nearly complete sealing is very difficult due to the complex shapes and structures formed when a prosthetic mitral valve body is deployed between the anterior leaflet and the posterior leaflet. This complexity is increased when the prosthetic valve has been engineered with features to accommodate LVOT problems and commissural leakage issues. Anterior leaflet prolapse, dislocations, pressure folds, leaflet eversion, leaflet inversion, and so forth are some of the problems that can arise with a prosthetic valve solution.

Functions of the Anterior Leaflet Clip Device

The anterior leaflet clip device functions by forming a pinched clip for attaching the native anterior leaflet to a prosthetic mitral valve. The clip device is attached to the prosthetic mitral valve body portion, either as an integral component of the valve body structure or as a separate deice that is sutured in place. The clip device is constructed to bend or conform such that it can compress the native anterior leaflet against the side wall of the valve body. The clip device is also constructed with a plurality of suture holes to facilitate a permanent joining of the anterior leaflet to the valve body. Attachment mechanisms other than sutures are also contemplated.

During systole the subannular space is filled and the anterior leaflet clip forms an additional seal against retrograde hemodynamic forces. During ventricular contraction or systole, the blood is ejected towards the prosthetic mitral valve. Retrograde blood hitting the prosthetic valve leaflets cause the leaflets to close, preventing regurgitation into the left atrium. Retrograde blood will then fill the subannular space around the chordae tendinae, which is frequently the cause and location of leakage around prosthetic valves which have been deployed into and through the native valve and annulus. However, the anterior leaflet clip device is constructed with a size and/or type of material so as to cause the retrograde blood to be blocked and avoid retrograde leaks.

### Functions of the Flared End of the Stent to Effect Atrial Sealing

The flared collar-end, also known as a collar or cuff, functions in a variety of ways. The first function of the flared end or cuff is to inhibit perivalvular leak/regurgitation of blood around the prosthesis. By flexing and sealing across the irregular contours of the annulus and atrium, leakage is minimized or prevented.

The second function of the flared end or cuff is to provide an adjustable and/or compliant bioprosthetic valve. The heart and its structures undergo complex conformational changes during the cardiac cycle. For example, the mitral valve annulus has a complex geometric shape known as a hyperbolic parobloid that is shaped like a saddle, with the horn being anterior, the seat back being posterior, and the left and right valleys located medially and laterally. Beyond this complexity, the area of the mitral annulus changes over the course of the cardiac cycle. Further, the geometry of the tricuspid valve and tricuspid annulus continues to be a topic of research, posing its own particular problems. Accordingly, compliance is a very important but unfortunately often overlooked requirement of cardiac devices. Compliance here refers to the ability of the valve to change conformation with the native annulus in order to maintain structural position and integrity throughout the cardiac cycle. Compliance with the motion of the heart is a particularly important feature, especially the ability to provide localized compliance where the underlying surfaces are acting differently from the adjacent surfaces. This ability to vary throughout the cardiac cycle allows the valve to remain seated and properly deployed in a manner not heretofore provided.

Additionally, compliance may be achieved through the use of the tethers where the tethers are preferably made from an elastic material. Tether-based compliance may be used alone, or in combination with the flared end or cuff-based compliance.

The third function of the flared end or cuff and valve is to provide a valve that, during implantation surgery, can contour to the irregular surfaces of the atrium. The use of independent tethers allows for side to side fitting of the valve within the annulus. For example, where three tethers are used, they are located circumferentially about 120 degrees relative to each other which allows the surgeon to observe whether or where perivalvular leaking might be occurring and to pull on one side or the other to create localized pressure and reduce or eliminate the leakage.

The fourth function of the flared end or cuff is to counter the forces that act to displace the prosthesis toward/into the ventricle (i.e. atrial pressure and flow-generated shear stress) during ventricular filling.

Additional features of the flared end or cuff include that it functions to strengthen the leaflet assembly/stent combination by providing additional structure. Further, during deployment, the flared end or cuff functions to guide the entire structure, the prosthetic valve, into place at the mitral annulus during deployment and to keep the valve in place once it is deployed. Another important function is to reduce pulmonary edema by improving atrial drainage.

### Flared End or Cuff Structure

The flared end or cuff is a substantially flat plate that projects beyond the diameter of the tubular stent to form a rim or border. As used herein, the term flared end, cuff, flange, collar, bonnet, apron, or skirting are considered to be functionally equivalent. When the tubular stent is pulled through the mitral valve aperture, the mitral annulus, by the tether loops in the direction of the left ventricle, the flared end or cuff acts as a collar to stop the tubular stent from traveling any further through the mitral valve aperture. The entire prosthetic valve is held by longitudinal forces between the flared end or cuff which is seated in the left atrium and mitral annulus, and the ventricular tethers attached to the left ventricle.

The flared end or cuff is formed from a stiff, flexible shape-memory material such as the nickel-titanium alloy material Nitinol® wire that is covered by stabilized tissue or other suitable biocompatible or synthetic material. In one embodiment, the flared end or cuff wire form is constructed from independent articulating radial tines or posts of wire extending axially around the circumference of the bend or seam where the flared end or cuff transitions to the tubular stent (in an integral flared end or cuff) or where the flared end or cuff is attached to the stent (where they are separate, but joined components).

Once covered by stabilized tissue or material, the articulating radial tines or posts of wire provide the flared end or cuff the ability to travel up and down, to articulate, along the longitudinal axis that runs through the center of the tubular stent. In other words, the individual articulating radial tines or posts of wire can independently move up and down, and can spring back to their original position due to the relative stiffness of the wire. The tissue or material that covers the flared end or cuff wire has a certain modulus of elasticity such that, when attached to the wire of the flared end or cuff, is able to allow the wire spindles to move. This flexibility gives the flared end or cuff, upon being deployed within a patient's heart, the ability to conform to the anatomical shape necessary for a particular application. In the example of a prosthetic mitral valve, the flared end or cuff is able to conform to the irregularities of the left atrium and shape of the mitral annulus, and to provide a tight seal against the atrial tissue adjacent the mitral annulus and the tissue within the mitral annulus. As stated previously, this feature importantly provides a degree of flexibility in sizing the a mitral valve and prevents blood from leaking around the implanted prosthetic heart valve.

An additional important aspect of the flared end or cuff dimension and shape is that, when fully seated and secured, the edge of the flared end or cuff preferably should not be oriented laterally into the atrial wall such that it can produce a penetrating or cutting action on the atrial wall.

In one preferred embodiment, the wire spindles of the flared end or cuff are substantially uniform in shape and size. In another preferred embodiment of the present invention, each loop or spindle may be of varying shapes and sizes. In this example, it is contemplated that the articulating radial tines or posts of wire may form a pattern of alternating large and small articulating radial tines or posts of wire, depending on where the valve is being deployed. In the case of a prosthetic mitral valve, pre-operative imaging may allow for customizing the structure of the flared end or cuff depending on a particular patient's anatomical geometry in the vicinity of the mitral annulus.

The flared end or cuff wire form is constructed so as to provide sufficient structural integrity to withstand the intracardiac forces without collapsing. The flared end or cuff wire form is preferably constructed of a superelastic metal, such as Nitinol® and is capable of maintaining its function as a sealing collar for the tubular stent while under longitudinal forces that might cause a structural deformation or valve displacement. It is contemplated as within the scope of the invention to optionally use other shape memory alloys such as Cu—Zn—Al—Ni alloys, and Cu—Al—Ni alloys. The heart is known to generate an average left atrial pressure between about 8 and 30 mm Hg (about 0.15 to 0.6 psi). This left atrial filling pressure is the expected approximate pressure that would be exerted in the direction of the left ventricle when the prosthesis is open against the outer face of the flared end or cuff as an anchoring force holding the flared end or cuff against the atrial tissue that is adjacent the mitral valve. The flared end or cuff counteracts this longitudinal pressure against the prosthesis in the direction of the left ventricle to keep the valve from being displaced or slipping into the ventricle. In contrast, left ventricular systolic pressure, normally about 120 mm Hg, exerts a force on the closed prosthesis in the direction of the left atrium. The tethers counteract this force and are used to maintain the valve position and withstand the ventricular force during ventricular contraction or systole. Accordingly, the flared end or cuff has sufficient structural integrity to provide the necessary tension against the tethers without being dislodged and pulled into the left ventricle. After a period of time, changes in the geometry of the heart and/or fibrous adhesion between prosthesis and surrounding cardiac tissues may assist or replace the function of the ventricular tethers in resisting longitudinal forces on the valve prosthesis during ventricular contraction.

### Stent Structure

Preferably, superelastic metal wire, such as Nitinol® wire, is used for the stent, for the inner wire-based leaflet assembly that is disposed within the stent, and for the flared end or cuff wire form. As stated, it is contemplated as within the scope of the invention to optionally use other shape memory alloys such as Cu—Zn—Al—Ni alloys, and Cu—Al—Ni alloys. It is contemplated that the stent may be constructed as a braided stent or as a laser cut stent. Such stents are available from any number of commercial manufacturers, such as Pulse Systems. Laser cut stents are preferably made from Nickel-Titanium (Nitinol®), but also without limitation made from stainless steel, cobalt chromium, titanium, and other functionally equivalent metals and alloys, or Pulse Systems braided stent that is shape-set by heat treating on a fixture or mandrel.

One key aspect of the stent design is that it be compressible and when released have the stated property that it return to its original (uncompressed) shape. This requirement limits the potential material selections to metals and plastics that have shape memory properties. With regards to metals, Nitinol® has been found to be especially useful since it can be processed to be austhenitic, martensitic or super elastic. Martensitic and super elastic alloys can be processed to demonstrate the required compression features.

### Laser Cut Stent

One possible construction of the stent envisions the laser cutting of a thin, isodiametric Nitinol tube. The laser cuts form regular cutouts in the thin Nitinol® tube. Secondarily the tube is placed on a mold of the desired shape, heated to the martensitic temperature and quenched. The treatment of the stent in this manner will form a stent or stent/flared end or cuff that has shape memory properties and will readily revert to the memory shape at the calibrated temperature.

### Braided Wire Stent

A stent can be constructed utilizing simple braiding techniques. Using a Nitinol® wire—for example a 0.012″ wire—and a simple braiding fixture, the wire is wound on the braiding fixture in a simple over-under braiding pattern until an isodiametric tube is formed from a single wire. The two loose ends of the wire are coupled using a stainless steel or Nitinol® coupling tube into which the loose ends are placed and crimped. Angular braids of approximately 60 degrees have been found to be particularly useful. Secondarily, the braided stent is placed on a shaping fixture and placed in a muffle furnace at a specified temperature to set the stent to the desired shape and to develop the martensitic or super elastic properties desired.

The stent as envisioned in one preferred embodiment is designed such that the ventricular aspect of the stent comes to 2-5 points onto which anchoring sutures are affixed. The anchoring sutures (tethers) will traverse the ventricle and ultimately be anchored to the epicardial surface of the heart approximately at the level of the apex. The tethers when installed under slight tension will serve to hold the valve in place, i.e. inhibit paravalvular leakage during systole.

### Leaflet and Assembly Structure

The valve leaflets are held by, or within, a leaflet assembly. In one preferred embodiment of the invention, the leaflet assembly comprises a leaflet wire support structure to which the leaflets are attached and the entire leaflet assembly is housed within the stent body. In this embodiment, the assembly is constructed of wire and stabilized tissue to form a suitable platform for attaching the leaflets. In this aspect, the wire and stabilized tissue allow for the leaflet structure to be compressed when the prosthetic valve is compressed within the deployment catheter, and to spring open into the proper functional shape when the prosthetic valve is opened during deployment. In this embodiment, the leaflet assembly may optionally be attached to and housed within a separate cylindrical liner made of stabilized tissue or material, and the liner is then attached to line the interior of the stent body.

In this embodiment, the leaflet wire support structure is constructed to have a collapsible/expandable geometry. In a preferred embodiment, the structure is a single piece of wire. The wireform is, in one embodiment, constructed from a shape memory alloy such as Nitinol®. The structure may optionally be made of a plurality of wires, including between 2 to 10 wires. Further, the geometry of the wire form is without limitation, and may optionally be a series of parabolic inverted collapsible arches to mimic the saddle-like shape of the native annulus when the leaflets are attached. Alternatively, it may optionally be constructed as collapsible concentric rings, or other similar geometric forms, each of which is able to collapse or compress, then expand back to its functional shape. In certain preferred embodiments, there may be 2, 3 or 4 arches. In another embodiment, closed circular or ellipsoid structure designs are contemplated. In another embodiment, the wire form may be an umbrella-type structure, or other similar unfold-and-lock-open designs. A preferred embodiment utilizes super elastic Nitinol® wire approximately 0.015″ in diameter. In this embodiment, the wire is wound around a shaping fixture in such a manner that 2-3 commissural posts are formed. The fixture containing the wrapped wire is placed in a muffle furnace at a pre-determined temperature to set the shape of the wire form and to impart it's super elastic properties. Secondarily, the loose ends of the wireform are joined with a stainless steel or Nitinol® tube and crimped to form a continuous shape. In another preferred embodiment, the commissural posts of the wireform are adjoined at their tips by a circular connecting ring, or halo, whose purpose is to minimize inward deflection of the post(s).

In another preferred embodiment, the leaflet assembly is constructed solely of stabilized tissue or other suitable material without a separate wire support structure. The leaflet assembly in this embodiment is also disposed within the lumen of the stent and is attached to the stent to provide a sealed joint between the leaflet assembly and the inner wall of the stent. By definition, it is contemplated within the scope of the invention that any structure made from stabilized tissue and/or wire(s) related to supporting the leaflets within the stent constitute a leaflet assembly.

In this embodiment, stabilized tissue or suitable material may also optionally be used as a liner for the inner wall of the stent and is considered part of the leaflet assembly.

Liner tissue or biocompatible material may be processed to have the same or different mechanical qualities, such as thickness, durability, etc., from the leaflet tissue.

Deployment within the Valvular Annulus

The prosthetic heart valve is, in one embodiment, apically delivered through the apex of the left ventricle of the heart using a catheter system. In one aspect of the apical delivery, the catheter system accesses the heart and pericardial space by intercostal delivery. In another delivery approach, the catheter system delivers the prosthetic heart valve using either an antegrade or retrograde delivery approach using a flexible catheter system, and without requiring the rigid tube system commonly used. In another embodiment, the catheter system accesses the heart via a trans-septal approach.

In one non-limiting preferred embodiment, the stent body extends into the ventricle about to the edge of the open mitral valve leaflets (approximately 25% of the distance between the annulus and the ventricular apex). The open native leaflets lay against the outside stent wall and parallel to the long axis of the stent (i.e. the stent holds the native mitral valve open).

In one non-limiting preferred embodiment, the diameter should approximately match the diameter of the mitral annulus. Optionally, the valve may be positioned to sit in the mitral annulus at a slight angle directed away from the aortic valve such that it is not obstructing flow through the aortic valve. Optionally, the outflow portion (bottom) of the stent should not be too close to the lateral wall of the ventricle or papillary muscle as this position may interfere with flow through the prosthesis. As these options relate to the tricuspid, the position of the tricuspid valve may be very similar to that of the mitral valve.

In another embodiment, the prosthetic valve is sized and configured for use in areas other than the mitral annulus, including, without limitation, the tricuspid valve between the right atrium and right ventricle. Alternative embodiments may optionally include variations to the flared end or cuff structure to accommodate deployment to the pulmonary valve between the right ventricle and pulmonary artery, and the aortic valve between the left ventricle and the aorta. In one embodiment, the prosthetic valve is optionally used as a venous backflow valve for the venous system, including without limitation the vena cava, femoral, subclavian, pulmonary, hepatic, renal and cardiac. In this aspect, the flared end or cuff feature is utilized to provide additional protection against leaking.

### Tethers

In one preferred embodiment, there are tethers attached to the prosthetic heart valve that extend to one or more tissue anchor locations within the heart. In one preferred embodiment, the tethers extend downward through the left ventricle, exiting the left ventricle at the apex of the heart to be fastened on the epicardial surface outside of the heart. Similar anchoring is contemplated herein as it regards the tricuspid, or other valve structure requiring a prosthetic. There may be from 1 to 8 tethers which are preferably attached to the stent.

In another preferred embodiment, the tethers may optionally be attached to the flared end or cuff to provide additional control over position, adjustment, and compliance. In this preferred embodiment, one or more tethers are optionally attached to the flared end or cuff, in addition to, or optionally, in place of, the tethers attached to the stent. By attaching to the flared end or cuff and/or the stent, an even higher degree of control over positioning, adjustment, and compliance is provided to the operator during deployment.

During deployment, the operator is able to adjust or customize the tethers to the correct length for a particular patient's anatomy. The tethers also allow the operator to tighten the flared end or cuff onto the tissue around the valvular annulus by pulling the tethers, which creates a leak-free seal.

In another preferred embodiment, the tethers are optionally anchored to other tissue locations depending on the particular application of the prosthetic heart valve. In the case of a mitral valve, or the tricuspid valve, there are optionally one or more tethers anchored to one or both papillary muscles, septum, and/or ventricular wall.

The tethers, in conjunction with the flared end or cuff, provide for a compliant valve which has heretofore not been available. The tethers are made from surgical-grade materials such as biocompatible polymer suture material. Non-limiting examples of such material include ultra high-molecular weight polyethylene (UHMWPE), 2-0 exPFTE (polytetrafluoroethylene) or 2-0 polypropylene. In one embodiment the tethers are inelastic. It is also contemplated that one or more of the tethers may optionally be elastic to provide an even further degree of compliance of the valve during the cardiac cycle. Upon being drawn to and through the apex of the heart, the tethers may be fastened by a suitable mechanism such as tying off to a pledget or similar adjustable button-type anchoring device to inhibit retraction of the tether back into the ventricle. It is also contemplated that the tethers might be bioresorbable/bioabsorbable and thereby provide temporary fixation until other types of fixation take hold such a biological fibrous adhesion between the tissues and prosthesis and/or radial compression from a reduction in the degree of heart chamber dilation.

Further, it is contemplated that the prosthetic heart valve may optionally be deployed with a combination of installation tethers and permanent tethers, attached to either the stent or flared end or cuff, or both, the installation tethers being removed after the valve is successfully deployed. It is also contemplated that combinations of inelastic and elastic tethers may optionally be used for deployment and to provide structural and positional compliance of the valve during the cardiac cycle.

### Pledget

In one embodiment, to control the potential tearing of tissue at the apical entry point of the delivery system, a circular, semi-circular, or multi-part pledget is employed. The pledget may be constructed from a semi-rigid material such as PFTE felt. Prior to puncturing of the apex by the delivery system, the felt is firmly attached to the heart such that the apex is centrally located. Secondarily, the delivery system is introduced through the central area, or orifice as it may be, of the pledget. Positioned and attached in this manner, the pledget acts to control any potential tearing at the apex.

### Tines/Barbs

In another embodiment the valve can be seated within the valvular annulus through the use of tines or barbs. These may be used in conjunction with, or in place of one or more tethers. The tines or barbs are located to provide attachment to adjacent tissue. In one preferred embodiment, the tines are optionally circumferentially located around the bend/transition area between the stent and the flared end or cuff. Such tines are forced into the annular tissue by mechanical means such as using a balloon catheter. In one non-limiting embodiment, the tines may optionally be semi-circular hooks that upon expansion of the stent body, pierce, rotate into, and hold annular tissue securely.

### Stabilized Tissue or Biocompatible Material

In one embodiment, it is contemplated that multiple types of tissue and biocompatible material may be used to cover the flared end or cuff, to form the valve leaflets, to form a wireless leaflet assembly, and/or to line both the inner and/or outer lateral walls of the stent. As stated previously, the leaflet component may be constructed solely from stabilized tissue, without using wire, to create a leaflet assembly and valve leaflets. In this aspect, the tissue-only leaflet component may be attached to the stent with or without the use of the wire form. In a preferred embodiment, there can be anywhere from 1, 2, 3 or 4 leaflets, or valve cusps.

It is contemplated that the tissue may be used to cover the inside of the stent body, the outside of the stent body, and the top and/or bottom side of the flared end or cuff wire form, or any combination thereof.

In one preferred embodiment, the tissue used herein is optionally a biological tissue and may be a chemically stabilized valve of an animal, such as a pig. In another preferred embodiment, the biological tissue is used to make leaflets that are sewn or attached to a metal frame. This tissue is chemically stabilized pericardial tissue of an animal, such as a cow (bovine pericardium) or sheep (ovine pericardium) or pig (porcine pericardium) or horse (equine pericardium).

Preferably, the tissue is bovine pericardial tissue. Examples of suitable tissue include that used in the products Duraguard®, Peri-Guard®, and Vascu-Guard®, all products currently used in surgical procedures, and which are marketed as being harvested generally from cattle less than 30 months old. Other patents and publications disclose the surgical use of harvested, biocompatible animal thin tissues suitable herein as biocompatible “jackets” or sleeves for implantable stents, including for example, U.S. Pat. No. 5,554,185 to Block, U.S. Pat. No. 7,108,717 to Design & Performance-Cyprus Limited disclosing a covered stent assembly, U.S. Pat. No. 6,440,164 to Scimed Life Systems, Inc. disclosing a bioprosthetic valve for implantation, and U.S. Pat. No. 5,336,616 to LifeCell Corporation discloses acellular collagen-based tissue matrix for transplantation.

In one preferred embodiment, the valve leaflets may optionally be made from a synthetic material such a polyurethane or polytetrafluoroethylene. Where a thin, durable synthetic material is contemplated, e.g. for covering the flared end or cuff, synthetic polymer materials such expanded polytetrafluoroethylene or polyester may optionally be used. Other suitable materials may optionally include thermoplastic polycarbonate urethane, polyether urethane, segmented polyether urethane, silicone polyether urethane, silicone-polycarbonate urethane, and ultra-high molecular weight polyethylene. Additional biocompatible polymers may optionally include polyolefins, elastomers, polyethylene-glycols, polyethersulphones, polysulphones, polyvinylpyrrolidones, polyvinylchlorides, other fluoropolymers, silicone polyesters, siloxane polymers and/or oligomers, and/or polylactones, and block co-polymers using the same.

In another embodiment, the valve leaflets may optionally have a surface that has been treated with (or reacted with) an anti-coagulant, such as, without limitation, immobilized heparin. Such currently available heparinized polymers are known and available to a person of ordinary skill in the art.

Alternatively, the valve leaflets may optionally be made from pericardial tissue or small intestine submucosal tissue.

### Manufacture of Ultra-Thin Stabilized Tissue

In a preferred embodiment, ultra-thin vapor-cross linked stabilized bioprosthetic or implant tissue material is contemplated. Tissue having a 0.003″ (0.0762 mm) to about 0.010″ (0.254 mm) may be made using a process comprising the steps of: (a) vapor cross-linking a pre-digested compressed tissue specimen by exposing the tissue specimen to a vapor of a cross-linking agent selected from the group consisting of aldehydes, epoxides, isocyanates, carbodiimides, isothiocyanates, glycidalethers, and acyl azides; and (b) chemically cross-linking the vapor-cross-linked tissue specimen by exposing the vapor-crosslinked tissue specimen to an aqueous crosslinking bath for a predetermined time, such crosslinking bath containing a liquid phase of a crosslinking agent selected from the group consisting of aldehydes, epoxides, isocyanates, carbodiimides, isothiocyanates, glycidalethers, and acyl azides.

Such tissue may be porcine, ovine, equine or bovine in origin and preferably the initial material is taken from a bovine animal 30 days old or less, although tissue from older animals is contemplated as within the scope of the invention. In one preferred embodiment, the tissue specimen is subjected to chemical dehydration/compression and mechanical compression before cross-linking.

Pre-digestion is provided by digesting a harvested, cleaned pericardial tissue in a solution containing a surfactant, such as 1% sodium laurel sulfate. The chemical dehydration/compression step comprises subjecting the tissue specimen to hyperosmotic salt solution. And, the mechanical compression may be performed by subjecting the tissue specimen to a roller apparatus capable of compressing the tissue specimen to a thickness ranging from about 0.003″ (0.0762 mm) to about 0.010″ (0.254 mm).

The animal collagen tissue specimen is then chemically cross-linked first by exposing the tissue to formaldehyde vapor for approximately 10 minutes, and second by immersing the tissue in a glutaraldehyde solution for two consecutive sessions of approximately 24 hours each.

### DESCRIPTION OF FIGURES

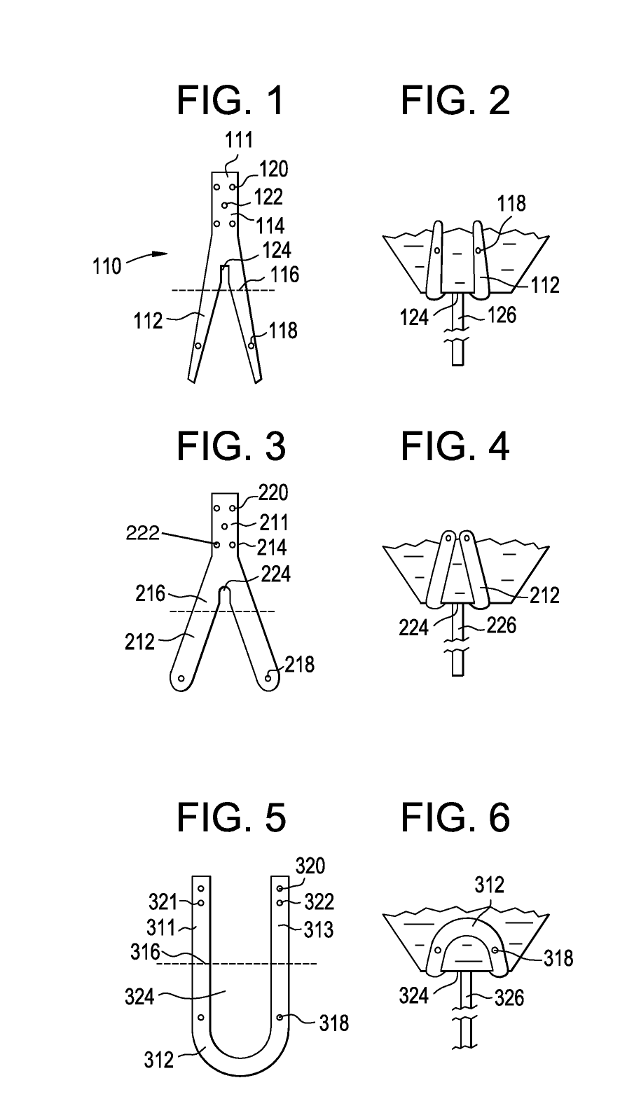

Referring now to the FIGURES, FIG. 1 is a side-view of one preferred type of anterior leaflet clip device 110 and shows an embodiment as a two-pronged hasp having a mounting segment 111, hinge segment 116, and leaflet capture and attachment segment 112. FIG. 1 shows stent mounting segment 114 with stent mounting suture holes 122. FIG. 1 also shows pre-aligned suture holes 120 in the mounting segment 111, and pre-aligned suture holes 118 in the leaflet capture and attachment segment 112. FIG. 1 shows valve tether aperture 124 as a mechanism to prevent an adjacent valve tether (not shown) from obstruct the clip device and to longitudinally traverse the clipping area while still providing for attachment of the anterior leaflet to the valve stent body.

FIG. 2 is a side view of the anterior leaflet clip device of FIG. 1 clipped onto a native anterior leaflet and shows valve tether 126 traversing through valve tether aperture 124.

FIG. 3 is a side-view of another preferred type of anterior leaflet clip device and shows an embodiment as a two-pronged hasp having a mounting segment 211, hinge segment 216, and leaflet capture and attachment segment 212. FIG. 3 shows stent mounting segment 214 with stent mounting suture holes 222. FIG. 3 also shows pre-aligned suture holes 220 in the mounting segment 211, and pre-aligned suture holes 218 in the leaflet capture and attachment segment 212. FIG. 3 shows valve tether aperture 224 as a mechanism to avoid obstructing and to accommodate the need of an adjacent valve tether (not shown) to not obstruct the clip device and to longitudinally traverse the clipping area while still providing for attachment of the anterior leaflet to the valve stent body.

FIG. 4 is a side view of the anterior leaflet clip device of FIG. 3 clipped onto a native anterior leaflet and shows valve tether 226 traversing through valve tether aperture 224.

FIG. 5 is a side-view of another preferred type of anterior leaflet clip device and shows an embodiment as a U-shaped hasp having a mounting segment 311, hinge segment 316, and leaflet capture and attachment segment 312. FIG. 5 shows stent mounting segments 311 and 313 with stent mounting suture holes 321 and 322. FIG. 5 also shows pre-aligned suture holes 320 in the mounting segment 311, and pre-aligned suture holes 318 in the leaflet capture and attachment segment 312. FIG. 5 shows valve tether aperture 324 as a mechanism to accommodate the need of an adjacent valve tether (not shown) to not obstruct the clip device and to longitudinally traverse the clipping area while still providing for attachment of the anterior leaflet to the valve stent body.

FIG. 6 is a side view of the anterior leaflet clip device of FIG. 5 clipped onto a native anterior leaflet and shows valve tether 326 traversing through valve tether aperture 324.

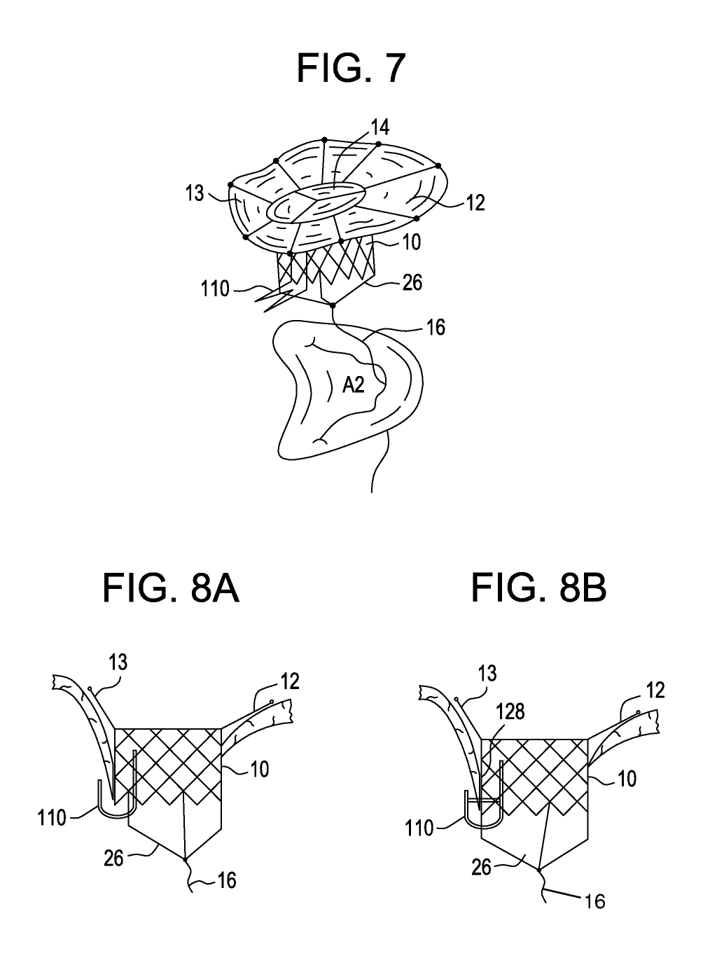

FIG. 7 is a perspective anterolateral view of one embodiment showing laser cut stent 10 with valve leaflets 14 mounted therein and with tissue-covered articulating collar structure 12 attached, and the anterior leaflet clip device 110 attached to the stent 10 and/or anterior collar 13, and providing an attachment mechanism to secure the anterior leaflet and provide a subvalvular seal against retrograde hemodynamic forces. FIG. 7 shows valve tether 26 connecting to apical ventricular tether 16. FIG. 7 is an atrial view after the valve is ejected from the delivery catheter but prior to the valve being seated into the mitral annulus and the consequential prosthetic valve deformation of the anterior leaflet A2 of the mitral valve.

FIG. 8A is a side of one embodiment showing laser cut stent 10 with valve leaflets mounted therein (not shown) and with tissue-covered articulating collar structure 12 attached, and the anterior leaflet clip device 110 attached to the stent 10, and providing an attachment mechanism to secure the anterior leaflet and provide a subvalvular seal against retrograde hemodynamic forces. FIG. 8A also shows the anterior collar segment 13 in the necessary angular geometry to avoid LVOT obstruction. FIG. 8A also shows valve tether 26 travelling unobstructed through the clip 110 and connecting with apical tether 16.

FIG. 8B is a side of one embodiment showing the leaflet clip 110 and suture 128 across the anterior leaflet and securing it to the laser cut stent body 10, providing an attachment mechanism to secure the anterior leaflet and provide a subvalvular seal against retrograde hemodynamic forces. FIG. 8B also shows the anterior collar segment 13 in the necessary angular geometry to avoid LVOT obstruction. FIG. 8B also shows valve tether 26 travelling unobstructed through the clip 110 and connecting with apical tether 16.

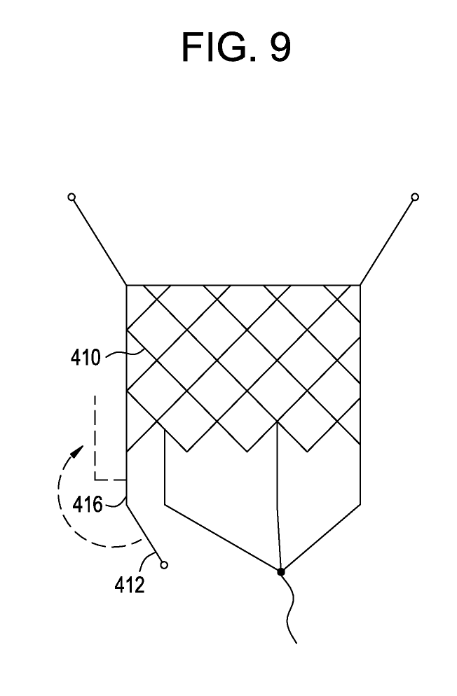

FIG. 9 is a side view of one embodiment showing the leaflet clip manufactured as an integral component of the laser-cut stent body 410. FIG. 9 shows how the hinge segment 416 and leaflet capture and attachment segment 412 move from the first deployment position to the second leaflet capture position.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

1. A pre-configured compressible transcatheter prosthetic cardiovascular valve having an anterior leaflet clip device, which comprises an expandable leaflet assembly comprised of stabilized tissue or synthetic material disposed within an expandable stent having at a distal end a plurality of articulating collar support structures having a tissue covering to form an atrial collar, said expandable stent having a proximal end comprised of an integral tether connection apparatus, said anterior leaflet clip device comprising a folding component that is mounted on the expandable stent, said folding component having a hinge segment and a leaflet capture and attachment segment.

2. The prosthetic heart valve of claim 1, further comprising wherein the anterior leaflet clip device is an integrated component of the expandable stent.

3. The prosthetic heart valve of claim 1, further comprising wherein the anterior leaflet clip device is a separate component that is sutured to the expanded stent before deployment of the valve, the device having a mounting segment connected to the hinge segment which is connected to the leaflet capture and attachment segment.

4. The anterior leaflet clip of claim 1, further comprising a plurality of pre-aligned suture holes in the leaflet capture and attachment segment.

5. The anterior leaflet clip of claim 3, further comprising a plurality of pre-aligned suture holes in the mounting segment and the leaflet capture and attachment segment wherein the suture holes line up in a substantially adjacent manner after the leaflet capture and attachment segment is folded over.

6. The prosthetic heart valve of claim 1, further comprising wherein the expandable stent has a low height to width profile.

7. The prosthetic heart valve of claim 1, further comprising wherein the expandable stent is a half-round D-shape in cross-section.

8. The prosthetic heart valve of claim 1, wherein the expandable stent is made from superelastic metal.

9. The prosthetic heart valve of claim 5, further comprising wherein the superelastic metal is a nickel-titanium alloy.

10. The prosthetic heart valve of claim 5, wherein the stent and cuff are formed from the same piece of superelastic metal.

11. The prosthetic heart valve of claim 1, further comprising wherein the expandable stent and collar are covered with stabilized tissue is derived from adult, 90-day old, or 30-day old, bovine, ovine, equine or porcine pericardium, or from animal small intestine submucosa.

12. The prosthetic heart valve of claim 1, further comprising wherein the expandable stent and collar are covered with synthetic material selected from the group consisting of polyester, polyurethane, polytetrafluoroethylene or another elastomeric material.

13. The prosthetic heart valve of claim 1, wherein the elastomeric material, stabilized tissue or synthetic material is treated with anticoagulant.

14. The prosthetic heart valve of claim 1, wherein the elastomeric material, the stabilized tissue or synthetic material is heparinized.

15. The prosthetic heart valve of claim 1, wherein integral tether connection apparatus has a plurality of tether attachment structures with anchoring tethers attached for anchoring the prosthetic heart valve to tissue.

16. The prosthetic heart valve of claim 15, wherein at least one of the plurality of tethers is a positioning tether and at least one of the plurality of tethers is an anchoring tether.

17. The prosthetic heart valve of claim 15, further comprising wherein one of the plurality of tethers is attached to an epicardial tether securing device.

18. The prosthetic heart valve of claim 1, wherein the valve does not use an anchoring tether or a positioning tether at all, and instead is held in the mitral annulus by the wrapping forces of the native leaflets, and optionally one or more standard anchoring elements, including but not limited to barbs, pins, and/or hooks, or combinations thereof.

19. A method of treating mitral regurgitation in a patient, which comprises the step of surgically deploying the prosthetic heart valve of claim 1 into the mitral annulus of the patient.

20. The method of claim 19, wherein the prosthetic heart valve is deployed by directly accessing the heart through the intercostal space, using an apical approach to enter the left ventricle, and deploying the prosthetic heart valve into the mitral annulus using a catheter delivery system.

21-27. (canceled)
